(19) United States
(12) Patent Application Publication
(10) Pub. No.: US 2020/0170507 A1
(43) Pub. Date: Jun. 4, 2020

(54) SYSTEM, DEVICE AND PROCESS FOR THE PERSONALISED HEALTH MONITORING OF ONE OR MORE PATIENTS AT RISK

(71) Applicant: EVOLUCARE TECHNOLOGIES, Villers Bretonneux (FR)

(72) Inventors: Alexandre LE GUILCHER, Garches (FR); Jerome GILBERT, Levallois Perret (FR)

(21) Appl. No.: 16/628,351

(22) PCT Filed: Jul. 2, 2018
(86) PCT No.: PCT/IB2018/054908

§ 371 Date: Jan. 3, 2020

(30) Foreign Application Priority Data
Jul. 5, 2017 (FR) 17/70716

(51) Int. Cl. A61B 5/00

(52) U.S. Cl. CPC A61B 5/002; A61B 5/7282; A61B 5/7275; A61B 5/0008; A61B 5/0022

(57) ABSTRACT

Disclosed is a system for the personalised health monitoring of one or more patients at risk of vital function failure, or at risk of relapse or the recurrence of a medical condition, with the aim of automatically and effectively generating appropriate alerts to health professionals. The system includes one or more units for supplying an information system with monitoring information, one or more personalisation data items specific to individual patients, an information system running personalised monitoring programmes for individual patients and one or more units for alerting one or more health professionals and/or one or more carers when an alert is generated. Also disclosed are a device and a process for the personalised health monitoring of one or more patients at risk of vital function failure, or at risk of relapse or the recurrence of a medical condition.

### TECHNICAL FIELD

This invention falls within the field of medical information devices and systems.

### PRIOR STATE OF THE ART

International studies have demonstrated abnormally high mortality rates in the majority of countries, following surgical procedures that do not present a particular risk. Other studies have established a correlation between the number of post-operative deaths per establishment and the number of nurses assigned to the monitoring of patients after they have been returned to their rooms. Further studies have shown that regular monitoring of the physiological, and even the psychological, state of patients suffering from a severe illness limited the probability of relapse, and the impact of such a relapse where relevant, since treatment could be provided at an earlier stage. In addition, there is economic pressure to reduce the numbers of nursing staff assigned to the monitoring of patients undergoing surgery and the monitoring of individuals who have been discharged from medical institutions, in order to increase the proportion of outpatient surgery within surgical procedures and generally to reduce the length of hospital stays. All these reasons call for technical solutions that will allow at least the partial automation of patient monitoring. Known solutions include those based on the use of hospital monitoring equipment, which are too costly and too invasive, those based on the use of mainstream ‘smart’ devices, which are not reliable enough, and those which use standard medical ‘smart’ devices, which are not specific or sensitive enough to meet the standards demanded by health professionals who wish to avoid false alarms without reducing their patients' chances.

### DISCLOSURE OF THE INVENTION

The purpose of this invention is to at least partially remedy the above-mentioned problems by proposing technical solutions that can be used in medical institutions and/or in patients' homes to monitor the health of individuals at risk, with the aim of automatically and effectively generating appropriate alerts.

According to its first aspect, this invention concerns a system for the personalised health monitoring of one or more patients at risk of vital function failure, or at risk of decompensation, or at risk of complications, or at risk of relapse or the recurrence of a serious medical condition such as stroke, heart attack or cancer, with the aim of automatically and effectively generating appropriate alerts to health professionals and/or carers. The system according to this invention is unique in that it comprises:
one or more means for supplying an information system with monitoring information; these means can be controlled by the said information system during monitoring, according to a patient's clinical condition and/or developments in this condition, and/or one or more of their responses to one or more previous questions. These means are, in addition, linked to a patient by means of a unique identifier. These means are controllable according to a patient's initial clinical condition and/or developments in this condition, i.e. controllable under the device's initial configuration so that its future operation will be appropriate to the medical context of a patient's clinical case but also, where relevant, so that the device can be controlled at any time during the monitoring of a particular patient. For example, in order to carry out measurements to verify one or more parameters or physiological manifestations of the failure of at least one vital function in a patient being monitored based on the results of previous measurements, or in order to ask more relevant questions by taking responses to previous questions into account, in accordance with predetermined rules implemented by software into this invention's monitoring system;
one or more personalisation data items specific to individual patients, which have been at least partly predetermined and/or validated in advance by medical intelligence. Since the aforementioned data items are linked to a patient by means of a unique identifier, the one or more personalisation data items are derived from the presetting of the monitoring system by a health professional and/or from the importing into the monitoring system according to this invention of external data concerning the patient to be monitored or at least one other patient presenting similarities to the patient to be monitored. This is to enhance operational responsiveness during monitoring within the unique context of individual patients and ultimately to enhance sensitivity and/or specificity when screening for anomalies which could pose a risk to a patient's vital functions, and would require urgent intervention by a health professional. Medical intelligence is defined as a medically competent human intelligence and/or an artificial intelligence that has been approved for medical use by an authority;
an information system running personalised monitoring programmes for individual patients, using data from the one or more means for supplying monitoring information, and based on at least one personalised data item, an alert being generated by the said information system if it interprets one or more conditions as being abnormal for the patient concerned. The concept of an information system in terms of this invention encompasses all technically relevant architectures, for example a centralised architecture where processing is executed entirely within a computer or within servers, or a more or less distributed architecture which comprises programmatic components executed within terminals and/or within all or part of the aforementioned means for supplying the information system with monitoring information;
one or more means for alerting one or more health professionals and/or one or more carers when an alert is generated by the said information system or by the one or more means supplying the information system with monitoring information. Depending on the implementation variants, these may consist of fixed alert means, for example a tablet or standard computer dedicated to the implementation of the system according to the invention, or a specific application executed by standard IT equipment which is also used for other purposes, or alternatively specific equipment dedicated to the implementation of this invention. Fixed alert means are to be conveniently located in places where a health professional is permanently on call, for example in a hospital nursing station. Provision is also made for the implementation of the invention using technical means such as intercom linked to a building. The invention's preferred implementation variants use mobile terminals to alert health professionals and/or carers. In general, carers are the first to be alerted in cases where patients are being monitored at home, for example following outpatient surgery. Depending on the implementation variants and the system's configuration according to the invention, the system alerts one or more carers via local alert means or via a previously registered telephone number. Where appropriate, the alerted carer is responsible for alerting a health professional or a medical emergency service. In other variants in the invention's implementation, the system alerts one or more carers so that they can provide human support but the system also simultaneously alerts a health professional who then makes the necessary medical arrangements, according to information concerning the patient's condition that has been transmitted via the monitoring system.

The system according to this invention is also unique in that at least one of the one or more personalisation data items specific to each patient relates to an adjustment and/or validation carried out by a health professional or an artificial intelligence approved for medical use by an authority, and/or is a data item that relates to at least one medical condition affecting the patient, and/or is a data item that relates to one or more items of information contained within a hospital information system, and/or is a data item that relates to at least one dose of medication taken by the patient, and/or is a data item that relates to a biological examination of the patient, and/or is a data item that relates to a radiological examination of the patient, and/or is a data item that relates to one of a patient's physical characteristics, and/or is a data item that relates to one of a patient's genetic characteristics or to his/her family history, and/or is data that relates to one or more so-called “omic” technologies. Since this data is obtained at multiple biological levels, from gene sequencing to the expression of proteins and metabolic structures, this data can cover all the mechanisms involved in the variations that occur in cellular networks and which influence the functioning of organic systems in their entirety.

The system according to this invention is unique in that the aforementioned information system also comprises one or more feedback loops operating one or more alert qualifications validated by health professionals or artificial intelligence approved for medical use by an authority, in order to automatically enhance the sensitivity and/or specificity of the monitoring. The invention makes provision for the use of feedback within the context of enhancing the monitoring of individual patients. The invention also provides for the use of feedback within the context of enhancing the monitoring of patients other than those for whom alerts have been medically validated. For example, by extracting data from the monitoring and/or monitoring settings of a patient whose alert has been validated by a doctor, and data that is characteristic of a given clinical situation or of a predetermined patient context and which is of use in the monitoring of other patients in similar clinical conditions or whose contexts share at least some common characteristics.

The system according to this invention is also unique in that the aforementioned information system comprises one or more software subassemblies that exploit data relating to patients being monitored, that has been obtained prior to the automated monitoring of their health. This enhances sensitivity and/or specificity in the detection of anomalies in individual patients by using previously obtained patient data to calibrate anomaly-detection algorithms and/or to set alert trigger thresholds that are above the values considered normal in their particular cases.

The system according to this invention is unique in that the aforementioned information system also comprises one or more predictive alert generating algorithms based on the exploitation of weak signals and/or data from the previous monitoring of patients presenting one or more similar characteristics. In general, the earlier a health problem is detected, the more effective its treatment. The invention thus makes provision for the exploitation of warning signs that are not yet significant enough in themselves to generate an alert but which, in the preliminary analysis of a very large number of patient cases, have been associated with a high probability of the subsequent occurrence of a problem, and justify a proactive alert.

The system according to this invention is unique in that the aforementioned information system also comprises a patient management software subassembly designed to facilitate the initial personalisation of a patient's monitoring by importing one or more of the system's initial setting parameters which are shared by patients with one or more characteristics in common. For example, within a patient profile management software subassembly, each profile is, for example, linked to a medical condition or common combination of medical conditions so that by simply selecting the name of the medical condition or common combination of medical conditions within a patient's records, an appropriate monitoring system setting—or combination of settings and appropriate functional options where relevant—is instantly predetermined. If necessary, more precise settings may be obtained using a basic automatic preset. This invention's refinements are advantageous in that they are time-saving for health professionals in charge of configuring the monitoring system for new patients, and significantly reduce the potential for error.

The system according to this invention is unique in that the aforementioned information system also comprises one or more computer processes which combine a patient's monitoring information, one or more personalisation data items specific to individual patients, and one or more rules specific to one or more medical conditions.

The system according to this invention is unique in that the aforementioned information system also comprises means for exporting and means for importing data representing knowledge acquired by the system during its operation, this data being associated with the system's configuration and to adjustments made to it, and/or with clinical profiles and/or with one or more rules specific to one or more medical conditions and/or with the system's predictive behaviour. This refinement in the invention is designed to enable the transmission of knowledge acquired by the monitoring system during its operation, for example in order to share this knowledge with similar, new or recently implemented systems. These system functionalities implemented by software may also be used to share and/or standardise experience gained by monitoring systems within individual hospital groups or to develop the knowledge acquired by an operator's monitoring system.

The system according to this invention is also unique in that the aforementioned information system comprises two or more monitoring software agents on the basis of one software agent per patient being monitored. These software agents are independent programme components that operate autonomously within their respective contextual environments to ensure that each patient has access to their own individual autonomous monitoring software entity.

The system according to this invention is also unique in that these monitoring software agents cooperate with each other so that any improvements in sensitivity and/or specificity in the detection of alert conditions obtained from software agents under given conditions, benefits at least one other software agent performing monitoring under at least partially similar conditions. This invention provides for the transfer of information between software agents for all types of at least partial similarities. For example, similarities in medical conditions or combinations of medical conditions, in one or more physiological parameters or in the physiological manifestations of vital function failure in patients being monitored, in the administration of medication that has identical active ingredients or produces the same effects, in patients' genetic backgrounds, histories, or predispositions, in clinical contexts, etc.

The system according to this invention is unique in that the aforementioned information system also comprises one or more artificial intelligence software subassemblies to enhance sensitivity and/or specificity during screening for one or more alert conditions. Within the context of the invention, artificial intelligence is defined as any software subassembly that imitates or replaces a human medical expert within certain implementations of their cognitive functions.

The system according to this invention is unique in that the aforementioned information system also comprises two or more artificial intelligence software subassemblies, presenting one or more diversities, with combined outputs in order to enhance sensitivity and/or specificity in the generation of alerts. In fact, in view of the limitations, functional specialisations and differences in performance of the algorithms used in artificial intelligence software subassemblies when faced with a given problem, this invention makes provision for the processing of complex and multifactorial patient monitoring issues by appropriately combining the results generated by one or more different artificial intelligence software subassemblies in order to obtain better results in combination than those that would be provided by individual artificial intelligence software subassemblies.

The system according to this invention is also unique in that this diversity in two or more artificial intelligences relates to the sources of the data being supplied to them, and/or the prerequisites for their implementation, and/or their operating principles, and/or their association with individual but similar patients and/or those with predetermined differences.

The system according to this invention is also unique in that, under the information system, alerts that are generated are accompanied by information characterising the degree of urgency of the response required by such alerts and/or informing health professionals about values and/or trends in all or part of the information involved in the generation of an alert. This refinement in the invention allows the health professionals who receive the alerts to decide between the various actions to be taken, based on their relative priorities, and to start thinking about the most appropriate care to be given to patients whilst patients await their arrival.

The system according to this invention is also unique in that, under this information system, alerts that are generated are accompanied by one or more instructions to be followed that are consistent with the cause of the alert. Instructions of this type, known to health professionals as “actions to be taken”, are intended to ensure that a patient receives prompt attention from the nearest health professionals when an alert is generated; such professionals may not necessarily possess all the medical expertise required but may be able to perform basic first aid on a patient prior to the arrival of other professionals with a higher level of expertise in relation to the clinical case of a patient concerned by an alert. This invention ensures that the nature and/or degree of the detail in the instructions given to the person receiving the alert corresponds to the technical characteristics of the terminal used. Depending on a terminal's retrieval capabilities, a brief text message may be displayed on a screen, for example, or an audio message or a video sequence may be played, etc. More elaborate implementation variants offer tutorials related to alerts, so as to enable low-skilled health workers to perform first aid or to prepare patients without putting them in danger prior to the arrival of more qualified health professionals. This refinement in the invention is also appropriate when the person alerted is a carer who may not necessarily possess the skills required to keep a patient safe prior to the arrival of a health professional.

The system according to this invention is also unique in that its one or more means for alerting one or more health professionals and/or one or more carers determine which health professional(s) and/or carer(s) are to be alerted, based on their physical distance from the patient concerned by the alert and/or the journey time involved in their reaching the patient and/or the availability of the said health professionals and/or carers at the time of the alert and/or criteria relating to the occupational competence of such health professionals. This refinement in the invention is designed to optimise the targeting of alerts by sending them to the professional or professionals best able to respond to them, through the use of related data such as, for example, the location of individual professionals who could potentially be alerted, the location of the patient concerned by the alert, and information regarding qualifications linked to the identifiers of health professionals within a database.

The system according to this invention is also unique in that its one or more means for alerting one or more health professionals comprises one or more software subassemblies for processing one or more acknowledgements from health professionals and/or carers who have responded to an alert. This applies, for example, to the acknowledgement of a response to an alert in cases where a system is designed to allow the interruption of audible alarms and/or escalation mechanisms during the search for an available health professional. Within certain implementation variants, this also applies to a second type of acknowledgement which allows health professionals who have responded to an alert to notify the system that the cause of the alert has been addressed. Within particularly sophisticated implementation variants of the invention, the system reactivates an alert if the anticipated acknowledgement(s) is/are not received within predetermined maximum time limits.

The system according to this invention is unique in that it also comprises one or more software subassemblies for guaranteeing the traceability and/or handling of alerts. This involves, for example, the continuous storage of data representing the timestamps of responses and/or, where relevant, acknowledgements of alerts and the identifiers of health professionals who have responded to alerts and/or the names of carers who have been declared as specific patients' potential carers.

The system according to this invention is also unique in that its one or more means for alerting one or more health professionals and/or one or more carers use a standard terminal with technical characteristics that allow it to be used for other purposes. “Standard” is the term used to describe electronic equipment with technical characteristics that have been designed to comply with the industry's technical standards, rather than to function exclusively within the system according to the invention, for example pager-type terminals, DECT-standard telephones connected to private networks, and cell phones or smartphones connected to standard public wireless networks, for example smartphones belonging to health professionals and carers that are used for personal or professional purposes. The system according to this invention has been designed to communicate directly with such standard terminals, or via a gateway or an appropriate subsystem located between the aforementioned information system according to the invention and a standard terminal.

The system according to this invention is also unique in that its one or more means for alerting one or more health professionals and/or one or more carers uses a terminal with one or more technical characteristics which are specific to the aforementioned system. These include, for example, software that is specific to the system according to the invention and run by a computer, a digital tablet or a standard market smartphone. Depending on the implementation variants, they also include electronic devices specific to the system according to the invention. The system according to the invention has been designed to communicate directly with such specific terminals, or via a gateway or an appropriate subsystem located between the said information system according to the invention and a specific terminal.

The system according to this invention is also unique in that one or more of its means for supplying an information system with monitoring information comprises one or more means for receiving responses from patients being monitored to questions that are put to them via a terminal, the software controlling this means for supplying an information system with monitoring information during monitoring being an adaptation of all or part of the questions put to patients, according to their clinical condition and/or its development, and/or at least one response to at least one previous question. This invention makes provision for several implementation variants so as to be able to offer optimal ergonomics to the main categories of patient users. For example, applications that can run on smartphones or standard tablets—for example under Android mobile operating systems (a trademark of Google, Inc.) or iOS (a trademark of Apple, Inc.)—have been designed to support patient-system interactions for categories of patients accustomed to using these devices. The functional richness of these devices makes it possible to envisage various ways of putting questions and receiving responses from patients, including in the form of games using visual animation for younger patients. For categories of patients who are less comfortable with mobile technology, there is provision for an online version that runs on standard PC or Mac microcomputers will be made available. For categories of patients who are resistant to the new technology, there is provision for a voice interface that can be used with all types of telephones. Speech synthesis software asks the questions and multi-speaker voice recognition software recognises simple responses based on anticipated key words. There is also provision for a simplified version that receives responses to questions when certain keys on a telephone keypad, proposed in the questions, are touched.

The system according to this invention is unique in that one or more of its means for supplying the aforementioned information system with monitoring information also comprises one or more ‘smart’ devices linked to this system. Such devices allow the system to receive information without the risk of its being altered by patients. This includes quantitative information associated with patients being monitored, such as their weight, blood pressure, temperature, etc. and qualitative information such as a physical activity indicator. This refinement offers the advantages of having to put fewer questions to patients and avoiding reading and typing errors and psychological bias in their responses. The connection between the ‘smart’ devices and the system according to the invention is frequently indirect, because current communicating devices use low-power, short-range wireless connections, in accordance with Bluetooth or Wi-Fi standards, for example. In these cases, communications are made via smartphones or tablets, via ADSL or fibre optic boxes or via a public or private infrastructure network access point.

The system according to this invention is also unique in that one or more of its means for supplying an information system with monitoring information comprises a communicating device, at least one element of which is positioned adjacent to or in contact with one or more patients in order to monitor one or more physiological parameters and/or one or more physiological manifestations of one or more vital function failures in patients being monitored, the said communicating device being remotely controllable by the aforementioned information system so that its operation can be adapted to the clinical conditions of the one or more patients being monitored and/or to developments in these conditions.

The system according to this invention is also unique in that one or more of its physiological parameters and/or one or more of the physiological manifestations of one or more vital function failures in patients being monitored consist of a set of two to six physiological parameters and/or predefined elementary physiological manifestations and/or environmental parameters and/or contextual information, used in combination to allow any alterations in the vital functions of individuals being monitored to be detected, following the appropriate local and/or remote IT processing of all or part of the data linked to the said parameters and/or physiological manifestations.

The system according to this invention is also unique in that all or part of the above-mentioned parameters or elementary physiological manifestations are extracted from physical measurements taken by sensors which are fewer in number than these parameters or physiological manifestations. In fact the invention makes provision for the advantageous use of sensors, mainly using appropriate signal processing software to allow a single sensor or a single acquisition subassembly to supply a number of parameters or elementary physiological manifestations. For example, an electrode assembly with interface electronics can at least supply a patient's heart rate and respiratory rate, and an optical sensor using two wavelengths can supply their pulse and pulsed oxygen saturation.

According to a second aspect, this invention concerns a device for the personalised health monitoring of one or more patients at risk of vital function failure, or at risk of decompensation, or at risk of complications, or at risk of a relapse or the recurrence of a medical condition. This invention makes provision for both sedentary devices, i.e. those that remain attached to the place where one or more patients are being treated or reside, and devices that are mobile, i.e. those that may at least partly be worn by patients when they move around within convalescent or residential facilities or even, in the case of certain implementation variants, outside predetermined locations. This device's mobile variants also offer the advantage of managing geolocation information so that patients may be located.

The device according to this invention is unique in that it comprises:
one or more sensors associated with the measurement of one or more physiological parameters and/or screening for one or more physiological manifestations of the failure of one or more vital functions in patients being monitored;
one or more data emission subassemblies for the transmission of monitoring and/or alert data to a remote information system, and/or directly to the terminals in order to alert one or more health professionals and/or one or more carers, and/or to an intermediate terminal control subsystem;
one or more data emission subassemblies for receiving, from a remote information system, one or more personalisation data items and/or one or more remote commands and/or data from one or more programmatic components executable by the device;
one or more digital processing subassemblies comprising one or more microprocessors, one or memories within which one or more programmes are stored, one or more random access memories allowing the execution of one or more programmes, and appropriate interfaces. These include, for example, interfaces with one or more sensors, and/or with one or more data emission subassemblies, and/or with one or more data reception subassemblies and/or with any other integrated function blocks, for example one or more buttons, one or more luminous indicators, a buzzer, a battery for monitoring the autonomy of the device, etc.;
one or more memories for storing one or more patients' unique identifiers and/or all or part of the one or more personalisation data items and/or data from the one or more programmatic components executable by the device. This removable or non-removable memory is included within the one or more digital processing subassemblies. Depending on the implementation variants, the one or more memories within the meaning of this invention are storage spaces dedicated to personalisation data, within one or more memories which are also used for other purposes within the one or more digital processing subassemblies, or they include one or more memories intended for this purpose;
one or more power supply subassemblies linked to an internal and/or external power source. Depending on the implementation variants, this power source is built in when mobility or an absence of wires is required or external when it is convenient to plug the monitoring device into the main electricity supply, or into a data communication network which also supplies power to connected terminals, or for example into a medical bed's autonomous power supply. Provision is made for variants comprising both an external power supply connection and an emergency in-built power supply for use in the event of an interruption to the external power supply.

The device according to this invention is also unique in that one or more of its sensors, one or more of its physiological parameters and one or more of its physiological manifestations of the failure of one or more of a monitored patient's vital functions, one or more of its data emission subassemblies, one or more of its data reception subassemblies, one or more of its personalisation data items, one or more of its remote commands, one or more of its programmatic components executable by the device, one or more of its digital processing subassemblies, one or more of its memories and one or more of its power supply subassemblies linked to an energy supply consist, respectively, of:
a temperature sensor in contact with the skin, a temperature sensor not in contact with the skin that uses the infrared radiation emitted by the body, a sensor in the form of two or more electrodes placed in contact with the skin that detects the electrical signals emitted by the body, a body impedance sensor in the form of two or more electrodes placed in contact with the skin, a mechanical deformation sensor in the form of elastic material with an electrical characteristic that varies with its elongation, an acoustic sensor maintained in contact with the body, an acoustic sensor placed away from the body, a two or more light wavelength relative absorption optical sensor, a two or more light wavelength relative reflection optical sensor, a sensor associated with artery compression, a Doppler velocity sensor, a motion sensor that is attached to a patient's body or limb—such as, for example, a multiple-axis accelerometer and/or a gyroscope for monitoring a patient's physical activity, an electronic communications device such as a smartphone that includes such sensors and that may be used by this device as a remote sensor subassembly, a camera with appropriate image analysis software, a brain wave sensor, environmental parameter sensors, and contextual information sensors;
body temperature, heart rate, respiratory rate, mechanical indications of the failure of one or more vital functions—for example respiratory failure—such as see-saw breathing or a drawing of the soft tissues below the level of the bones supporting them, stress in accessory respiratory muscles, venous oxygen saturation, blood pressure, cardiac output, one or more clinical signs of decompensation such as abnormally pale skin, cyanosis of the extremities, one or more clinical signs of complications, excessive perspiration, convulsions or abnormal movements, abnormal clinical manifestations of the central nervous system, abnormal reactions to mechanical, electrical or light stimulation, vocal emissions such as one or more cries or groans, verbal responses to a request, pain assessment;
a one-way radio frequency transmitter, or a two-way radio frequency transmitter, an online carrier current modem used as a one-way transmitter or a two-way online carrier current transmitter, an interface for connecting a cable or an optical fibre, an interface for transmitting data that uses single- or multiple-wavelength light rays that are visible or invisible to the human eye, an interface with control electronics for near- or far-field electrical, capacitive, inductive, magnetic, electromagnetic or acoustic coupling, this coupling being two-way or one-way in a device-to-infrastructure direction;
a one-way radio frequency receiver, or a two-way radio frequency transmitter, an online carrier current modem used as a one-way receiver or a two-way online carrier current transmitter, an interface for connecting a cable or an optical fibre, an interface for extracting data from single or multiple-wavelength light rays that are visible or invisible to the human eye—for example an infrared signal receiver or a receiver for visible light that has been modulated according to the standard known as “Li-Fi”, for example; an interface with control electronics for near- or far-field electrical, capacitive, inductive, magnetic, electromagnetic or acoustic coupling, this coupling being two-way or one-way in a device-to-infrastructure direction. Examples include electrical, capacitive and inductive coupling, coupling via audio signals such as infrasound, audible sound or ultrasound, via electromagnetic waves, for example via NFC-type means or using a means for recharging an internal induction accumulator, for example by demodulating the operating frequency of the inductor in order to extract data;
one or more addresses in a data communication network, one or more device identifiers, one or more patient identifiers associated with the said device, one or more data items relating to an adjustment to the functioning of the said device concerning a patient associated with that device;
a measurement or read request concerning the status of one of the device's sensors or functional subassemblies. For example, the voltage of a battery supplying power to all or part of the said device or the index of a running time counter in order to estimate the remaining running time of the device, a regular measurement or read request concerning the status of one of the device's sensors or functional subassemblies, the matching data being transmitted by the device at a predetermined frequency or under predetermined conditions;
a script, an applet, an application, built-in software. For example, in the case of an at least partly decentralised architecture, in order to execute all or part of the monitoring processes including the rules concerning the generation of alerts from one or more measurement values and/or a status value from one or more of the device's sensors. The invention also makes provision for the remote updating of all or part of the device's built-in software;
a microcontroller, an electronic component known as a “system on a chip”, or a standard modular subassembly. For example a standard PC-compatible processing card that conforms with the PC/104 standard for example, a Raspberry Pi card (a trademark of Raspberry Pi Foundation), an Arduino card (a trademark of Arduino Srl) or any other standard card, module or component that functions as a digital processing subassembly and has the computing power and interfaces required for the implementation of the device according to the invention;
a volatile or non-volatile memory included in an electronic component known as a “system on a chip”, in a component or functional subassembly dedicated to data storage, or in a removable medium such as a smartcard, SD memory card, USB stick or any functional equivalent or successor to these standard removable memory media;
an electronic power supply subassembly combined with one or more rechargeable accumulators, one or more batteries, one or more supercapacitors, a wireless near- or far-field energy receiver, a PoE Ethernet connection, an AC or DC power supply subassembly or a power supply subassembly connected to one or more external power sources. For example, the battery in an autonomous medical bed or one or more energy sensors, for example photovoltaic cells conveniently combined with artificial lighting that can also transmit data, for example by using communication techniques known as “VLC” or “Li-Fi”.

The device according to this invention is unique in that it also comprises an emergency call control that can be operated by one or more of the patients being monitored. Examples include a manually operated “pear” or pendant-type wired or wireless control equipped with a push button, a button that can be activated by patients and which is located on one or more of the wearable parts of the device, a control that uses gesture or image recognition or voice recognition in calls for help and/or any other control device adapted to accommodate a particular form of handicap.

The device according to this invention is also unique in that it comprises a basic subassembly and one or more acquisition subassemblies including the one or more sensors associated with the measurement of one or more physiological parameters and/or screening for one or more physiological manifestations of the failure of one or more vital functions in patients being monitored; this basic subassembly and the one or more acquisition subassemblies communicate via wired and wireless means. For example, the basic subassembly and the one or more acquisition subassemblies communicate simultaneously or alternately via a two-way radio frequency or wired link. Provision is made for economical implementation variants of the invention within which, for example, the basic subassembly transmits personalisation information to the one or more acquisition subassemblies prior to entering the monitoring phase, for example by using means such as inductive coupling which is also used to charge an accumulator within such an acquisition subassembly, the latter transmitting data collected via a one-way radio frequency or infrared link during the monitoring phase to the said basic subassembly.

The device according to this invention is also unique in that the above-mentioned basic subassembly comprises a means for charging a rechargeable power supply contained within the one or more acquisition subassemblies. Where a basic subassembly is designed to control several acquisition subassemblies, the said base is also designed to be able to charge acquisition subassemblies supplied with batteries either simultaneously or in sequence, depending on the implementation variant.

The device according to this invention is also unique in that the above-mentioned basic subassembly comprises a means to control all or part of the one or more acquisition subassemblies' functions.

The device according to this invention is also unique in that the above-mentioned basic subassembly comprises a means to simultaneously manage the monitoring of a plurality of patients within the same location or within neighbouring locations within range of the means of communication operating between the said basic subassembly and the acquisition subassemblies concerned. Neighbouring locations are defined as being within the same room or within adjacent rooms separated by one or more walls, or within separate individual cubicles within a single treatment room, or at individual locations within a recovery room, or within a temporary field hospital- or crisis cell-type structure, or in an ambulance-type vehicle.

The device according to this invention is also unique in that the above-mentioned basic subassembly comprises a means to alert one or more health professionals and/or one or more carers when an alert is generated. The invention conveniently makes provision for the basic subassembly according to the invention to also comprise a means such as an acoustic alarm or a radio frequency transmitter capable of communicating directly with terminals such as the pagers worn by health professionals within a medical facility. Provision is also made for variants within which the basic subassembly comprises a means of connecting with a local telecommunications infrastructure, allowing standard terminals such as cordless DECT telephones to be used, or a means of connecting with remote telecom infrastructures allowing standard cell phones to be used.

The device according to this invention is also unique in that the above-mentioned basic subassembly and/or acquisition subassembly comprise a means of signalling and/or acknowledging an alert that has been generated on behalf of the one or more patients with one or more acquisition subassemblies attached to the said basic subassembly. For example, this may consist of a luminous indicator positioned in a highly visible area of the device, for signalling that an alert has been generated on behalf of a patient—or one of the patients—attached to the said basic subassembly. This also makes it easier to locate patients in distress in treatment rooms where there are a number of devices according to the invention. There is also provision for a basic subassembly comprising a means—such as, for example, a push button located on top of it—that acknowledges an alert when the person alerted arrives at a patient's bedside. There is also provision, within variants in which the basic subassembly supervises several acquisition subassemblies for the inclusion of a means of signalling that allows health professionals to quickly locate a patient for whom an alert has been generated amongst the patients being monitored.

The device according to this invention is also unique in that both it and the system according to the invention in general treat any technical anomaly that may affect patient monitoring as a reason to generate an alert. Examples include hardware or software failure or a shortage in the power being supplied to all or part of the device, a connection failure that is liable to prevent the transmission of data that is liable to generate an alert, etc. Provision is made for refinements to the invention, such as distinguishing between the causes of technical alerts and the causes of medical alerts so that, respectively, only individuals involved in the technical maintenance of the monitoring system or health professionals involved in caring for patients will be alerted.

The device according to this invention is also unique in that one or more of its acquisition subassemblies comprise a means for transmitting information relating to their energy autonomy and/or a means to adapt all or part of their functions to the amount of power remaining in their self-contained power supplies. An acquisition subassembly's self-contained power supply may be insufficient and this potentially constitutes a risk that alerts will not be generated. Depending on the implementation variants, the invention provides for the continuous monitoring of the energy autonomy of the device's relevant subassemblies, and can even extend their autonomy by adopting strategies to reduce power consumption whilst still ensuring a level of service that is sufficient to keep patients out of danger. For example, by spacing out—or even halting—the most energy-consuming measurements, such as the pulsed oxygen saturation measurement where this is implemented using optoelectronic means.

According to a third aspect, this invention concerns a process that allows the automatic and efficient generation of alerts to health professionals and/or carers by the system according to the invention for the personalised health monitoring of a plurality of patients.

The process according to the invention is unique in that it comprises these phases:
the initial adjustment of one or more of the operating parameters of the means for supplying an information system with monitoring information and/or of the conditions for the implementation of the personalised monitoring programme, based on one or more items of information specific to the patients being monitored;
the regular measurement of one or more physiological parameters and/or screening for one or more physiological manifestations of the failure of one or more vital functions in patients being monitored and/or the receipt of one or more responses to one or more questions put to patients being monitored and/or the receipt of one or more data items transmitted by a ‘smart’ device. The nature and frequency of the said measurements and/or, where relevant, the sampling of screening results is determined by criteria associated with the medical conditions of the patients being monitored, for example, and/or by their clinical conditions and/or by developments in these conditions and/or, where necessary, by the energy autonomy of the means for supplying an information system with monitoring information;
the determination by the said information system and/or by the means for supplying monitoring information, as to whether or not an alert should be generated, based on at least one result from at least one measurement of at least one physiological parameter and/or at least one screening for one or more physiological manifestations of the failure of one or more vital functions in a patient being monitored and/or at least one response to one or more questions put to a patient being monitored and/or at least one data item transmitted by a ‘smart’ device, and based on at least one item of information specific to a patient being monitored. This phase, which is implemented by software, is implemented according to partial or complete architectural variants within the said information system and/or within the one or more means for supplying an information system with monitoring information;
the alerting of one or more health professionals and/or one or more carers when an alert is generated by the said information system or by the one or more means for supplying monitoring information.

The process according to the invention is unique in that it also comprises an adjustment phase for one or more operating parameters in the means for supplying an information system with monitoring information and/or in the conditions for the implementation of the personalised monitoring programme, based on developments in the clinical condition of patients being monitored. This phase, which is implemented by software, is implemented according to partial or complete architectural variants within the said information system and/or within the one or more means for supplying an information system with monitoring information.

The process according to this invention is unique in that it also comprises a phase for the prior acquisition of one or more items of reference information. For example, before treatment or clinical events prior to monitoring. This phase of the process according to the invention allows the personalised calibration of the system according to the invention, for example by acquiring all or part of the one or more physiological parameters prior to medical procedures that justify the monitoring of patients. In practice, this phase of the process according to the invention is conveniently arranged to take place during medical examinations preceding scheduled procedures—for example, in the case of surgical procedures, during a prior appointment with an anaesthetist. This phase, reduced to the rapid acquisition of the one or more physiological parameters which will be monitored by the system according to the invention following a medical procedure, may also conveniently be included in cases of unscheduled medical procedures, for example as part of emergency medical procedures, or even in field medicine where patient flow can be very high and where a post-operative monitoring system is particularly suited to dealing with peak situations where nursing staff numbers may rapidly become insufficient for the effective monitoring of patients receiving treatment.

The process according to this invention is unique in that it also comprises a phase for managing the acknowledgement and/or non-acknowledgement of alerts within a predetermined time by the one or more health professionals and/or carers who have been alerted. This phase, which is implemented by software, is executed according to partial or complete architectural variants within the aforementioned means for supplying the information system with monitoring information. The invention makes provision for the traceability of interventions by identifying the one or more health professionals and/or one or more carers who treated—or attempted to treat—a patient, and recording the corresponding time-stamped event. Provision is also made for refinements that reactivate alerts and, where necessary, redirect so-called escalation mechanisms towards higher levels within an organisation if the anticipated acknowledgement(s) are not received within a predetermined period.

Using the process according to the invention to monitor the health of one or more patients after they have been returned to their rooms following a medical procedure in a medical institution. For example, after leaving the operating theatre or recovery room following surgery or after leaving a resuscitation unit, and up until the end of the critical period following the procedure or until the patient is discharged from the medical institution.

Using the process according to the invention to monitor the health of one or more patients who have undergone outpatient surgery where there is a risk of complications, whilst one or more of these patients is/are under observation following their return home or within a follow-up care facility or convalescent institution or within a residential home for the dependent elderly.

Using the process according to the invention to monitor the health of one or more patients in a recovery room or in an intensive care unit, following surgery.

Using the process according to the invention to monitor the health of one or more patients at risk of relapse or the recurrence of a condition. In fact the invention allows early alerts to be generated, allowing patients to be treated more rapidly and therefore, depending on the medical condition, improving patients' vital prognoses or limiting the effects of relapse or such recurrence.

Using the process according to the invention to monitor the health of one or more animals following veterinary treatment. The invention is not, in fact, reserved for the automated health monitoring of one or more human patients. The technical solutions provided by the invention are directly transferable to veterinary care, since skilled health professionals may be specialists in either medicine, surgery or animal care.

### BRIEF DESCRIPTION OF THE DRAWINGS

The invention's other advantages and characteristics will become evident upon examination of the detailed description of the implementation methods—which are by no means exclusive—and accompanying drawings in which:

FIG. 1 shows the structure of the system according to the invention.

FIG. 2 shows the first variant of the system according to the invention.

FIG. 3 shows the second variant of the system according to the invention.

FIG. 4 shows the first variant using artificial intelligence.

FIG. 5 shows the combining of artificial intelligence programmes.

FIG. 6 shows the structure of the device according to the invention.

FIG. 7 shows the first variant of the device according to the invention.

FIG. 8 shows the second variant of the device according to the invention.

FIG. 9 shows the process according to the invention.

### DETAILED DESCRIPTION OF THE FIGURES AND IMPLEMENTATION METHODS

The invention's other features and further advantages will become evident in the description below and also in the attached drawings given as examples, which are by no means exclusive:

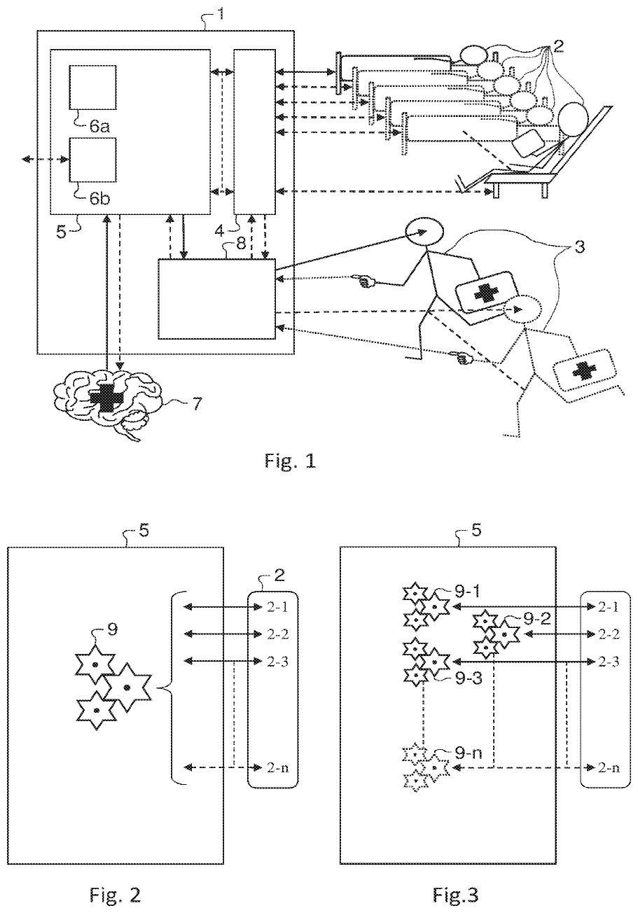

FIG. 1 shows the structure of the system according to the invention. The structure of the system according to the invention 1 for the personalised monitoring of a patient's vital functions 2 for the automatic and effective generation of appropriate alerts to health professionals and/or carers 3 comprises one or more means 4 for supplying an information system with monitoring information 5. These one or more means 4 can be controlled by the said information system 5 during monitoring, according to individual patients' clinical conditions 2 and/or developments in these conditions, and/or one or more of their responses to one or more previous questions. This invention makes provision for means 4 that are based on questions put to patients and that include, where relevant, data that has been automatically supplied to the information system 5 via ‘smart’ devices. The means 4 consist, for example, of one or more computer programmes or software components, their means of implementation and a means of presenting them to patients in order to generate questions that are specific to individual patients. Means of presenting questions to patients consist, for example, of a digital tablet, a smartphone, a television with a direct connection or one that is connected via an adaptor, a telephone, a watch, a bracelet but in addition, in the case of the invention's universal variant, a device that also permits the automatic acquisition of monitoring information. The aforementioned ‘smart’ devices consist, for example, of ‘smart’ scales for monitoring changes in a patient's weight, a ‘smart’ blood pressure monitor that monitors blood pressure or a ‘smart’ electronic medication organiser to monitor adherence to a prescribed treatment without requiring a patient's input. This variant of the invention, which is based on adaptive questionnaires and, where relevant, on the patients' use of small communicating devices to facilitate the monitoring of their health, requires the possession of cognitive abilities. Should this not be the case, or where a patient prefers a variant in which he/she plays a passive role, regarding the means 4 the invention makes provision for the use of a controllable device that permits the automatic acquisition of monitoring information. The invention provides that at least one of the means 4 should be a combination of the system's two main variants, comprising either one or more automatic controllable devices for monitoring a patient's health, and progressive questionnaires supplemented, where necessary, by data supplied via third-party devices.

The system according to the invention also comprises one or more personalisation data items 6a, 6b specific to individual patients. These one or more personalisation data items have been at least partly predetermined and/or validated beforehand by human or artificial medical intelligence 7. These one or more personalisation data items 6a result from a system-specific setting or configuration carried out or prescribed by a health professional or by medical artificial intelligence that has been approved by the authorities, and are based on information about patients to be monitored with which they are familiar, for example following consultations and/or after reading reports, biological analysis results, imaging results, etc. Provision has also been made for deriving one or more personalisation data items 6a from calibration carried out prior to the clinical events justifying a patient's monitoring, for example prior to surgery. Depending on the implementation variants for the means 4 for supplying monitoring information, the one or more personalisation data items consist of one or more of their responses to one or more relevant questions and/or one or more reference monitoring data items derived from the one or more means 4 according to the invention. Provision has also been made for obtaining one or more items of personalisation data 6b from sources outside the system according to the invention, for example from hospital information systems or shared external patient records. Provision has also been made within the system according to the invention for combining internal data resulting from prior configurations, or from calibrations involving patients, with one or more data items derived from external sources, in order to refine and/or safely manage the personalisation of the monitoring. The automatic monitoring system according to the invention also comprises an information system 5 that executes a personalised monitoring programme for individual patients, using data derived from the one or more means for supplying monitoring information 4, and based on one or more personalisation data items 6a, 6b. An alert is generated by the aforementioned information system if it interprets at least one condition as being abnormal for the patient concerned. The system according to the invention also comprises one or more means 8 for alerting one or more health professionals and/or one or more carers 3 when an alert is generated by the said information system 5. Provision is made for implementation variants that are decentralised from the system according to the invention, within which alerts are generated by the one or more means 4 for supplying monitoring information. Provision is also made for implementation variants within which the one or more means 4 for supplying monitoring information also comprise one or more means 8 for alerting one or more health professionals and/or one or more carers 3 when an alert is generated.

FIG. 2 shows the first variant of the system according to the invention. This implementation variant is based on a conventional information system within which one or more computer programmes or one or more software components 9 associated with a predefined function manage this function for all 2 the patients 2-1 to 2-n to be monitored.

FIG. 3 shows the second variant of the system according to the invention. This implementation variant is based on an information system within which individual patients to be monitored 2-1 to 2-n are each supported by one or more individual software components 9-1 to 9-n which include the execution environments required for the autonomous management of their patients. Autonomous programmatic entities 9-1 to 9-n assigned to given patients being monitored 2-1 to 2-n are known as monitoring software agents.

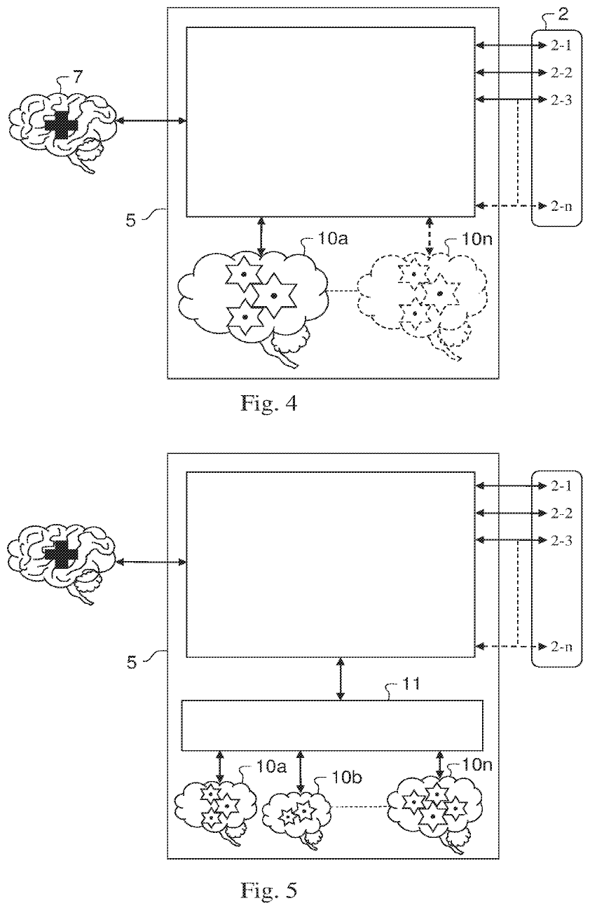

FIG. 4 shows the first variant using artificial intelligence within the information system 5. are provided Under this information system 5, provision is made for one or more software programmes known as artificial intelligence software programmes 10a to 10n, in order to enhance the performance of the automated patient monitoring system 2, in particular in terms of its sensitivity and/or specificity. These programmes complement conventional rules engine-type algorithms which determine whether an alert should be generated according to a medical condition, monitoring information and personalisation data such as data resulting from an initial configuration of the system by a medical intelligence 7 for a given patient 2-1 to 2-n. Provision is made for input from software packages with high logic complexity or algorithmic complexity that implement cognitive functions similar to those of humans—known as artificial intelligence software—in order, for example, to detect correlations in massive quantities of data derived from multiple patients that are difficult to detect using conventional means, to search for evidence or warning signs of ongoing physiological processes that may lead to proven vital failure in a given patient, and to fine-tune the configuration and personalisation of alert detection by automatically exploring data with weak links to the physiological processes being monitored—such as, for example, data representative of patients' backgrounds, histories, or genetic predispositions, etc.

FIG. 5 shows the combining of artificial intelligence programmes. This example of implementation differs from that shown in FIG. 4 in that the information system 5 comprises one or more software subassemblies 11 which combine the results of a plurality of artificial intelligence software programmes 10a to 10n based on different principles and/or different data sets, in order to produce superior combined results on one or more qualitative or quantitative criteria, for example to reduce the risk of error, enhance the sensitivity of screening or regarding the computing power required to process patients' 2-1 to 2-n data flows within an acceptable length of time, in contrast to the results that could be supplied by individual software programmes 10a to 10n. Provision is also made for combining the approaches shown in FIGS. 4 and 5, by implementing one or more specialised artificial intelligence software programmes that run alone in order to carry out tasks that obtain results that are considered satisfactory, and artificial intelligence software programmes accompanied by a software subassembly that combines results in order to carry out other tasks that require superior performances compared to those that can be achieved without being combined.

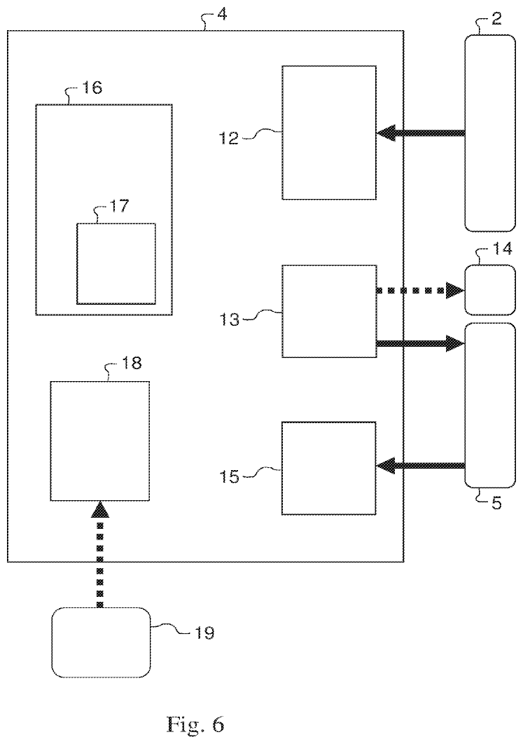

FIG. 6 shows the structure of the device according to the invention. The aforementioned device forms part of the means 4 according to the invention for supplying the information system with personalised monitoring information concerning the health of one or more patients 2 to 5. The device according to the invention comprises one or more sensors 12 associated with the measurement of one or more physiological parameters and/or screening for one or more physiological manifestations of the failure of one or more vital functions in the one or more patients being monitored 2. The device also comprises one or more data emission subassemblies 13 for the transmission of monitoring and/or alert data to a remote information system 5 and/or directly or indirectly to the terminals in order to alert one or more health professionals and/or one or more carers. The device also comprises one or more data reception subassemblies 15 for receiving, from a remote information system 5, one or more items of personalisation data and/or one or more remote commands and/or data from one or more programmatic components executable by the device. The device also comprises one or more digital processing subassemblies 16, for example an energy-efficient system on a chip comprising one or more architecture 32 or bit 64 microprocessors licensed by the company ARM Ltd., one or memories within which one or more programmes are stored, one or more random access memories allowing the execution of one or more programmes, an interface with the one or more captors 12, an interface with the one or more data emission subassemblies 13, an interface with the one or more data reception subassemblies 15, one or more memories 17 for storing one or more patients' unique identifiers and/or all or part of the one or more personalisation data items and/or data from the one or more programmatic components executable by the device. The device also comprises one or more power supply subassemblies 18 linked to an internal power source, for example a Li-ion battery or a battery with sufficient capacity to enable the device according to the invention to continuously and autonomously monitor a patient over 5 to 10 days. Where batteries are used, an electrical connection or inductive coupling, for example, is provided, with an external power source 19 such as a low voltage adaptor that can be connected to the power supply in order to charge the built-in battery between two periods of patient monitoring or during the continued monitoring of a single patient.

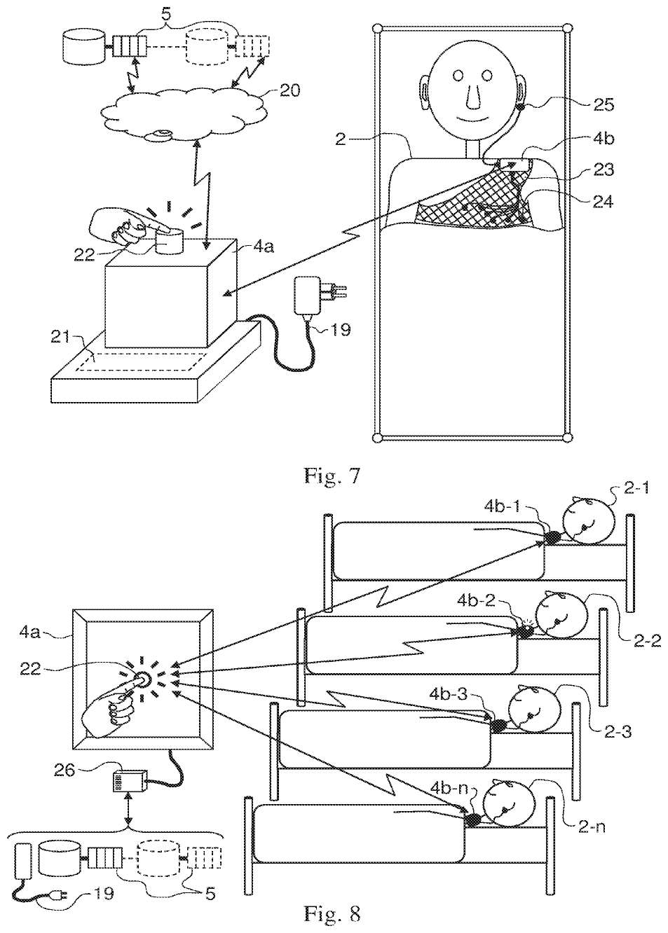

FIG. 7 shows the first variant of the device according to the invention. This is the first variant of the device according to the invention, which is based on the distribution of means and implemented functions between a basic subassembly and one or more acquisition subassemblies comprising the one or more sensors associated with the measurement of one or more physiological parameters and/or the screening for one or more physiological manifestations of the failure of one or more vital functions in a patient being monitored. This distribution across several subassemblies is mainly intended to optimise the device according to the invention in order to make it more compact and increase the autonomy of the element which is in contact with or in the vicinity of a patient. Another advantage of this distribution of the device's resources is to facilitate logistics and optimise the distribution of the cost of the equipment through the use of sterile single-use subassemblies, reconditionable where possible, for the equipment which is in contact with the patient. In this initial example, which is by no means exclusive, the device according to the invention is split into a base 4a that may be placed on a bedside table, for example, and the element 4b that is in contact with the patient 2. Each of these subassemblies comprises a digital processing subassembly and one or more suitable communication interfaces. The base communicates with one or more remote information systems 5 via a link to a telecommunications network 20. Depending on the implementation variants or the user's choices, this link may be direct, via a built-in radio modem which is compatible with a cellular network, or indirect via an Ethernet link provided in the form of a standard RJ45 connector or a Wi-Fi connection that can be established with a private access point, such as an ADSL or fibre optic box, or with a public access point. In addition, the base is connected to an external power source 19, since it combines the most energy-consuming technical subassemblies of the device according to the invention. A charging area 21 is also provided on the base, for charging the acquisition subassembly if necessary, by induction or via a direct electrical connection. The base also comprises a visible and easily-accessible luminous button. This luminous button is intended to be used to signal, via a green light, that the device is functioning at a nominal level, for example, and that the connections between the sensor subassembly and the base, and between the base and the remote information system, are functioning and that the power being supplied to the two autonomous subassemblies is sufficient. This button can also be used for the localised signalling of an alert via a flashing red light, and there is also provision for an audible alert to accompany this signal. Pressing the button acknowledges an alert and signals to the system that a patient is being attended to. The event is time stamped and recorded. During monitoring, pressing the button is interpreted as a call for help and a manual alert is generated. All events are time stamped and recorded. To avoid errors in manipulation or inadvertent pressure on a button, for example whilst rooms are being cleaned, the button must be held down for several seconds in order to trigger an alert. Of course using separate means for the signal and control functions still comes within the framework of the invention. In this by no means exclusive example, the sensor subassembly is in the form of a small case 4b that has been designed to be fixed to a single-use stretch fabric vest 23 in order that it should remain attached to a patient's body 2. Provision is made for several sensor subassembly and location variants, for example a bracelet, belt, pendant, self-adhesive patch, etc. The variant in FIG. 7 is preferable because it is well suited for extended use; its contact with the skin is not occlusive, the choice of a suitably elastic and quick-drying fabric ensures that the sensors remain attached, the skin can breathe and a patient can be washed without removing the device. Its position close to the left shoulder is compatible with all postures—lying, sitting and standing, patient discomfort is minimal and at least part of the device remains visible above the sheet when a patient is in bed. In addition, this location is particularly convenient in that it allows a number of sensors to be built in, minimising cable length where required due to its proximity to the essential vital organs. In the variant presented, an appropriate number of electrodes 24, for example between 2 and 6, are prepositioned at specific locations within the support vest that allow the heart and respiratory rates to be monitored. Pulsed oxygen saturation is monitored by means of a detachable sensor 25 mounted on a clip and fastened to the earlobe, which is less of a handicap to patients than the end of a finger. Provision is made for even more sophisticated variants that contain an built-in contact microphone facing the ribcage and acting as an electronic stethoscope, a body temperature sensor, a manual “SOS” alert button accessible to the patient, a microphone directed towards the patient's mouth and an audio transducer directed towards his/her ear in order, if necessary, to establish a phonic link with a remote health professional in the event of an alert. The two-way link between the basic subassembly and the one or more acquisition subassemblies conveniently uses low-energy radio connectivity with sufficient range; the standard known as “Bluetooth Low Energy” and “Bluetooth Smart” is particularly suitable for use according to the invention in terms of its electricity consumption, its range, its safety, the availability of its bit rates and due to the fact that many ‘smart’ devices use this standard and can therefore be used with this invention without requiring further developments. Of course other standards, for example low-energy variants of the Wi-Fi standard or proprietary solutions, may also be used within the framework of the invention. The miniature case 4b, which is conveniently waterproof so that it can be reused after disinfection, and so that patients can wash safely, is attached, depending on the variant, to the support vest using “snap” type fasteners, velcro patches (a trademark of the Velcro BVBA Plc company), or inserted into a special opening in the fabric support. The invention stipulates that only certain models of stretch-fabric vests can accommodate all patient morphologies and sizes, both male and female, and also those of animals, within the context of the invention's provision for veterinary use.

FIG. 8 shows the second variant of the device according to the invention. This variant differs from that shown in FIG. 7 in that it is optimised for routine use within a medical institution. The base 4a is designed for the supervision of a plurality of patients 2-1 to 2-n occupying a shared room or a recovery room, for example 2, 4 or more patients. The base 4a is designed as a flat case optimised for attachment to a wall. It is connected to one or more information systems 5 by means of an Ethernet connection that complies with the standard known as PoE (Power over the Ethernet), for example. Thus the same compact connector 26 that complies with the RJ45 standard, for example, or a connector for directly connecting the wires of such a connection, allows the basic subassembly 4a both to communicate with the one or more information systems 5 and to locate its power supply 19 within a building's local network infrastructure. Provision is also made for variants that communicate via Wi-Fi and are powered by a low-voltage adaptor that can be connected to an institution's emergency power supply, in order to meet all requirements. In addition to the indicator light 22 fitted to the basic subassembly for signalling an alert involving one or more of the patients being monitored who are connected to this base, the case 4b-1 to 4b-n in this variant of the device also comprises an indicator which flashes very brightly when the patient wearing it has generated an alert. This is so that when a health professional who has been notified by the system rushes into the (treatment) room concerned, he/she is able to immediately locate the patient 4b-2 with the problem from amongst the other patients being monitored, and attend to him/her without losing time and therefore without reducing the patient's chances. Provision has been made for charging the batteries within the acquisition subassemblies between uses by means of a charger which is not shown in the drawing. Consisting of an optimised implementation variant for monitoring patients within medical institutions, the charger is conveniently designed to simultaneously charge a plurality of acquisition subassemblies according to the invention, for example from 2 to 10 devices at the same time. Even more conveniently, in terms of a medical institution's productivity and the control of hospital-acquired infections, the charger combines the simultaneous charging by induction of a plurality of water-resistant devices, with their immersion in a disinfectant solution for the period of time required, so that the devices are both fully charged and completely disinfected. The charger is conveniently fitted with a device for detecting whether disinfectant liquid levels are sufficient, thus preventing their being charged and returned to service if they have not been properly disinfected.

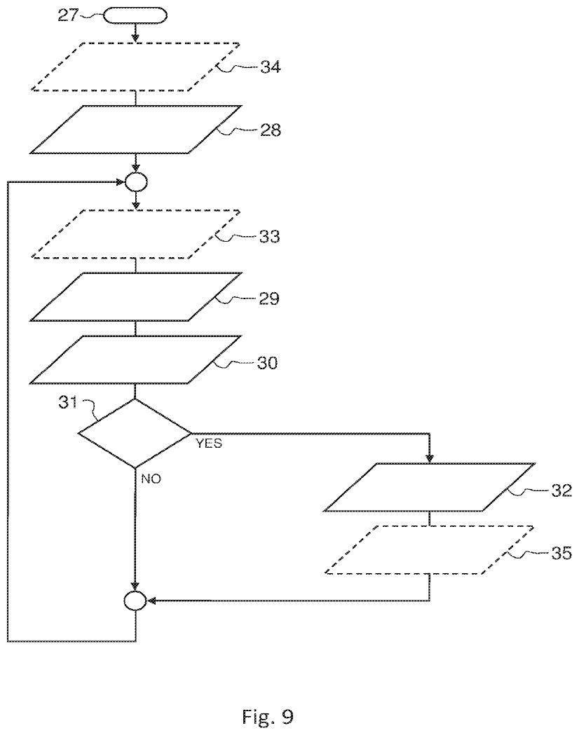

FIG. 9 shows the process according to the invention. Following the initialisation phase 27 at the powering-up of the IT equipment implementing the process, the process according to the invention commences with the phase 28 comprising the initial configuration of one or more of the operating parameters of the means for supplying the information system with monitoring information and/or the conditions for running the personalised monitoring programme, based on one or more items of information specific to the patient being monitored. This is followed by phase 29 which contains the regular measurement of one or more physiological parameters and/or screenings for one or more physiological manifestations of the failure of one or more vital functions in the patient being monitored and/or the reception of one or more responses to one or more questions put to the patient being monitored and/or the reception of one or more items of data transmitted by a ‘smart’ device. This is then followed by the phase 30 in which the aforementioned information system, and/or the one or more means for supplying the information system with monitoring information, determine whether an alert should be generated, based on one or more results from one or more measurements of one or more physiological parameters and/or one or more screenings for one or more physiological manifestations of the failure of one or more vital functions in the patient being monitored and/or one or more responses to one or more questions put to the patient being monitored and/or one or more data items transmitted by a ‘smart’ device, and on one or more items of information specific to the patient being monitored. If an alert is generated, the test 31 leads to phase 32 when one or more health professionals and/or one or more carers are alerted. In the absence of an alert, the phases of the process are run in a loop throughout the monitoring, starting from phase 29 or phase 33 where applicable. In certain implementation variants, the process according to the invention also comprises a phase 33 for adjusting one or more of the operating parameters of the means for supplying the information system with monitoring information and/or the conditions for running the personalised monitoring programme, according to changes in the clinical condition of the patient being monitored. In certain implementation variants, the process according to the invention also comprises a phase 34 for the prior acquisition of one or more items of reference information. In certain implementation variants, the process according to the invention also comprises a phase 35 for managing the acknowledgement and/or non-acknowledgement of alerts within predetermined time limits by the one or more health professionals and/or one or more carers alerted.

Of course, this invention is not limited to the examples just described and a number of modifications could be made whilst remaining within the framework of the invention, in particular by using technical standards that differ from those cited as examples, by combining several variants within the same implementation, by combining elements taken from various examples in different ways or by changing the order of the process's phases.

1-45. (canceled)

46. A system for personalised health monitoring of a patient at risk of at least one among vital function failure, decompensation, complications, relapse and recurrence of a medical condition, with the aim of automatically and effectively generating appropriate alerts to health professionals, wherein said system comprises:
at least one computer module for supplying a monitoring information to an information system concerning at least one of a physiological parameter, a physiological manifestation of a failure of at least one vital function in a patient being monitored and a response of a patient being monitored to questions that are asked to said patient via a terminal, said at least one computer module being controlled by said information system during monitoring, according to a clinical condition of the patient, developments in a clinical condition of the patient and one or more of said responses;
personalisation data items specific to individual patients, said personalisation data items having at least partly been predetermined and validated beforehand by a medical intelligence;
the information system running a personalised monitoring programme for individual patients, using data from the at least one computer module and based on the personalisation data items, an alert being generated by the information system if at least one condition of the patient is interpreted as being abnormal; and
an alert transmitter for alerting at least one health professional when an alert is generated by said information system.

47. The system according to claim 46, wherein the personalisation data items specific to individual patients relate to at least one of an adjustment and a validation carried out by a health professional and an artificial intelligence approved for medical use by an authority and a data item that relates to at least one medical condition affecting a patient and a data item that relates to one or more items of information contained within a hospital information system and a data item that relates to at least one dose of medication taken by a patient and a data item that relates to a biological examination of a patient and a data item that relates to a radiological examination of a patient and a data item that relates to physical characteristics of a patient and a data item that relates to a genetic characteristic of a patient or his/her family history and data that relates to one or more “omic” technologies.

48. The system according to claim 46, wherein the information system comprises one or more computing processors which combine a monitoring information of the patient, one or more personalisation data items specific to individual patients and one or more rules specific to one or more medical conditions.

49. The system according to claim 46, wherein the aforementioned information system comprises an output interface for exporting, and an input interface, for importing data representing knowledge acquired by the system during an operation of the system, said data being associated with at least one of a configuration of the system and adjustments made to the system and with clinical profiles and with one or more rules specific to one or more medical conditions and with a predictive behaviour of the system.

50. The system according to claim 46, wherein the at least one computer module for supplying monitoring information comprises one or more receivers for receiving responses from patients being monitored to questions that are asked to them via a terminal, a software controlling the at least one computer module for supplying monitoring information during monitoring being an adaptation of all or part of the questions asked to the patient according to at least one of a clinical condition of the patient and developments in a clinical condition of the patient, and at least one response to at least one of said questions.

51. The system according to claim 46, wherein the at least one computer module for supplying monitoring information to the information system further comprises at least one smart device connected to the system.

52. The system according to claim 46, wherein the at least one computer module for supplying monitoring information to the information system comprises a communicating device, at least a part of said communicating device being positioned adjacent to or in contact with one or more patients in order to monitor at least one of the physiological parameters and the physiological manifestations of one or more vital function failures in patients being monitored, said communicating device being remotely controllable by the said information system in order to adapt its functioning to the clinical conditions of the one or more patients being monitored and to developments in the clinical conditions.

53. The system according to claim 46, wherein the physiological parameters and the physiological manifestations of one or more vital function failures in patients being monitored consist of a set of two to six physiological parameters and predefined elementary physiological manifestations and environmental parameters and contextual information, used in combination to allow an alteration in the vital functions of individuals being monitored to be detected.

54. The system according to claim 46, wherein at least one of the physiological parameters and the physiological manifestations are extracted from physical measurements taken by sensors which are fewer in number than the physiological parameters or the physiological manifestations.

55. A method for automatically and effectively generating appropriate alerts to health professionals or carers by a system for personalised health monitoring of a plurality of patients according to claim 46, the method comprising:
initially adjusting one or more operating parameters of at least one of the at least one computer module for supplying monitoring information to the information system and the conditions for the implementation of the personalised monitoring programme, based on one or more items of information specific to the patient being monitored;
regularly measuring the physiological parameters and screening for at least one of the physiological manifestations of the failure of one or more vital functions in patients being monitored and a receipt of one or more responses to one or more questions asked to the patients being monitored and a receipt of one or more data items transmitted by a smart device;
determining by at least one of the information system and the at least one computer module for supplying monitoring information as to whether or not an alert should be generated, based on at least one result from at least one measurement of the physiological parameters and the screening for at least one of the physiological manifestations of the failure of one or more vital functions in patients being monitored and the at one or more responses to the one or more questions asked to the patients being monitored and the one or more data items transmitted by the ‘smart’ device, and based on at least one item of information specific to patients being monitored;
alerting of the health professionals when the alert is generated by the information system.

56. The method according to claim 55, wherein the method further comprises adjusting at least one operating parameter of the at least one computer module for supplying monitoring information to an information system and conditions for implementing the personalised monitoring programme based on developments in the clinical condition of the patient being monitored.

57. The method according to claim 55, wherein the method further comprises preliminary acquiring one or more items of reference information.

58. The method according to claim 55, wherein the method further comprises at least one of an acknowledgement and a non-acknowledgement within a predetermined time of the alert by at least one of the alerted one or more health professionals and the carers.
